(19) United States
(12) Patent Application Publication
(10) Pub. No.: US 2003/0008275 A1
(43) Pub. Date: Jan. 9, 2003

(54) Attenuated HIV strains and use thereof

(76) Inventors: Jaap Goudsmit, Amsterdam (NL); Marion Cornelissen, Mijdrecht (NL)

Correspondence Address: TRASK BRITT, P.O. BOX 2550, SALT LAKE CITY, UT, 84110, US

(21) Appl. No.: 09/948,977

(22) Filed: Sep. 7, 2001

Related U.S. Application Data
(60) Provisional application No. 60/231,067, filed on Sep. 8, 2000.

(51) Int. Cl. C12Q 1/70; A61K 39/21; C12N 7/01; A61K 38/04; C07H 21/04; C12Q 1/68; C12N 7/00; C07K 5/00; C07K 7/00; C07K 16/00; C07K 17/00

(52) U.S. Cl. 435/5; 435/235.1; 424/208.1; 435/6; 536/23.72; 530/328

(57) ABSTRACT

The present invention provides an isolated human immunodeficiency virus, and methods for obtaining the same, comprising at least one non-revertant mutation capable of delaying or diminishing the pathological behavior of said human immunodeficiency virus in comparison with a human immunodeficiency virus not having at least one such mutation. The invention further provides a vaccine against acquired immunodeficiency syndrome comprising the isolated human immunodeficiency virus according to the invention. Said virus can also be used for diagnostic assays in HIV-infected patients.

### CROSS-REFERENCE TO RELATED APPLICATION

Pursuant to the provisions of 35 U.S.C. §119(e), this application claims the benefit of the filing date of Provisional Patent Application Serial No. 60/231,067, filed Sep. 8, 2000, for “Attenuated HIV Strains and Use Thereof,” which is hereby incorporated herein by this reference.

### TECHNICAL FIELD

The present invention relates to the field of immunology, in particular to viruses and more in particular to human immunodeficiency virus.

### BACKGROUND

Live attenuated virus vaccines have been enormously successful. They are widely used to prevent diseases such as polio and measles. Until now, however, there has been no vaccine against acquired immunodeficiency syndrome (AIDS). All over the world, much research is being done with human immunodeficiency virus to obtain a suitable vaccine against AIDS. Although attenuated strains have been obtained, many safety concerns still remain about either the reversion of attenuated vaccine strains to virulent phenotypes or the induction of fulminant infection in immunocompromised individuals. An example of the possibility of attenuated strains to regain their pathological behavior is described in a recent publication by Berkhout et al. They demonstrated that the HIV-1 delta3 vaccine candidate, which contains 3 deletions in non-essential parts of the genome, is able to regain full replication capacity within four months of replication in tissue culture (Berkhout et al., 1999). Another proof of the genetic instability of attenuated strains is the finding by Baba et al. that deletion variants of the simian immunodeficiency virus (SIV) showed an increased ability to replicate after several years in some infected monkeys, concomitant with the onset of AIDS (Baba et al., 1999). Furthermore, some individuals who received a vaccine comprising attenuated HIV-1 variants lacking the nef gene recently showed a decline in CD4+ T-cell numbers, indicating that these individuals could develop AIDS (Dyer et al., 1999; Greenough et al., 1999). Thus, to date there is no suitable vaccine with live attenuated HIV. This kind of vaccine is to be preferred, however, because other vaccines comprising inactivated viruses or subunits do not result in a broad-based immune response or long-term memory necessary to confer life-long protection in immunized individuals. Therefore, live attenuated HIV vaccines are still under investigation.

### BRIEF SUMMARY OF THE INVENTION

The present invention relates to the unexpected and important finding that certain non-revertant mutations in a human immunodeficiency virus are capable of delaying or diminishing the pathological behavior of the virus for a very long time in vivo. We have isolated and sequenced such mutant human immunodeficiency viruses, which were derived with informed consent from a patient who lacks the characteristic decline in CD4+ T cell number. The individual that carried the HIV virus with the mutations described in Tables 1 through 4 was relatively healthy with high CD4+ cell counts in the blood. This phenomenon is uncommon in HIV infection, where a significant drop in the CD4+ cell count is normally observed. In this respect, it seemed the HIV virus that infected the patient was less- or even non-pathogenic. The HIV virus was, however, immunogenic as shown by the seroconversion of the individual. Furthermore, experiments with strains of the virus in vitro showed a normal growing pattern compared to human immunodeficiency viruses not having at least one such mutation (FIG. 2). These are suitable characteristics for a virus suited for vaccine development as a live attenuated vaccine. FIG. 1 shows the detected amount of HIV-RNA and CD4+ T cells in the patient during the last five years.

### BRIEF DESCRIPTION OF THE DRAWINGS

FIG. 1. The detected amount of HIV RNA and CD4+ T cells in a patient that carried HIV viruses with the mutations described in Tables 1 through 4.

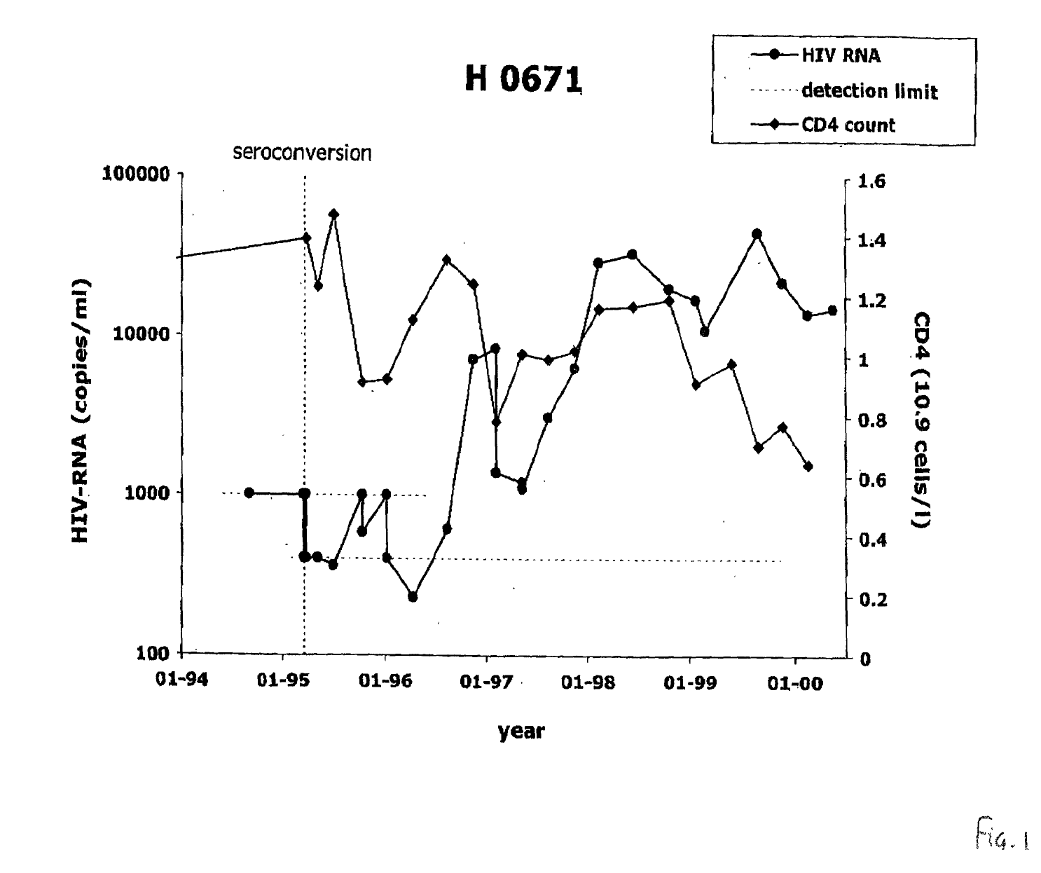

FIG. 2. Growing pattern in vitro of viruses with the mutations described in Tables 1 through 4.

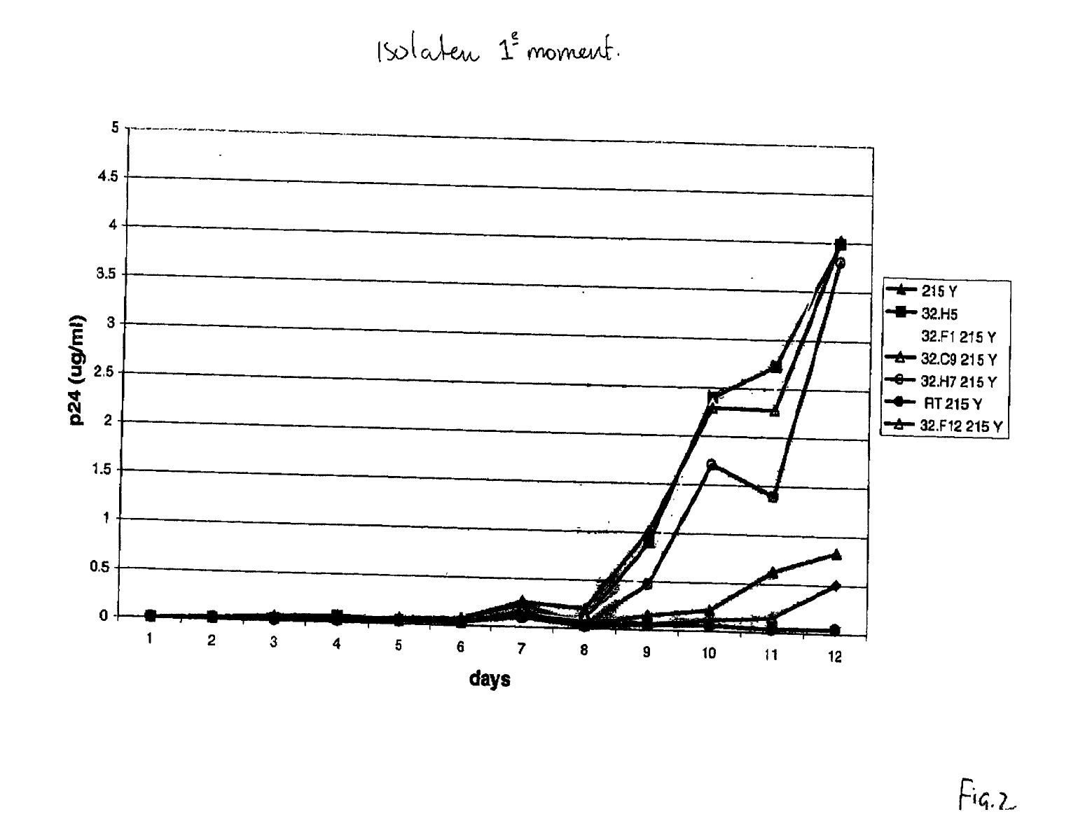

### DETAILED DESCRIPTION OF THE INVENTION

The present invention provides an isolated human immunodeficiency virus comprising at least one non-revertant mutation capable of delaying or diminishing the pathological behavior of the human immunodeficiency virus in comparison with a human immunodeficiency virus not having at least one such mutation. Preferably, the virus according to the invention is an HIV-1 virus.

A “non-revertant mutation” is defined as a mutation that is stable and remains present in the virus over a prolonged period of time. Preferably, the non-revertant mutation is stable and remains present in the virus over a prolonged period of time in vivo.

“Delaying the pathological behavior of the virus” means that it takes a longer time after primary infection before the amount of CD4+ T cells in the infected individual starts to decline as compared with the time it takes with a human immunodeficiency virus not having at least one such mutation.

“Diminishing the pathological behavior of the virus” is defined as decreasing the capability of the virus to significantly reduce the number of CD4+ T cells in an individual infected with the virus.

“Significantly reducing” is defined as reducing the number of CD4+ T cells to a greater extent than that which occurs during a normal variation within the individual.

“Substitution amino acid” is defined as an amino acid that does not substantially alter the capability of the amino acid sequence to delay or diminish the pathological behavior of the virus according to the invention as compared to a human immunodeficiency virus not having at least one such mutation.

Preferably, the virus according to the invention comprises at least one amino acid sequence as is described in Tables 1 and 2. Thus, in one embodiment, the invention provides a virus comprising at least one amino acid sequence as described in Tables 1 or 2. In another embodiment, the invention provides a virus comprising at least one amino acid sequence as described in Table 1. In a preferred embodiment, the invention provides an isolated virus according to the invention, wherein at least one of the non-revertant mutations is located in the gag or pol gene. Important mutations are the 3 amino acid (QAE) and 10 amino acid (QSRPEPTAPP) (SEQ ID NO: 1) insertions, the 2 amino acid deletion in the gag gene, and the “IPIK” mutation in the pol gene.

Alternatively, the virus of the invention may comprise at least one substitution amino acid in an amino acid sequence as described in Table 1 or 2. Thus, in another embodiment the invention provides a virus that comprises at least one substitution amino acid in at least one amino acid sequence as described in Table 1 or 2.

The virus according to the invention is obtainable by state-of-the-art cloning techniques. Those of ordinary skill in the art know a variety of ways to perform site-directed mutagenesis. Thus, the present invention also provides a method for obtaining the virus according to the invention comprising providing a wild-type human immunodeficiency virus with at least one non-revertant mutation capable of delaying or diminishing the pathological behavior of the human immunodeficiency virus in comparison with a human immunodeficiecy virus not having at least one such mutation.

Alternatively, the virus strain according to the invention can be isolatedbyrandomly collecting human immunodeficiency strains and selecting for strains comprising sequence similarities to the virus according to the invention.

“Sequence similarity” means that an isolated strain comprises at least one mutation in common with the virus according to the invention, the mutation being capable of delaying or diminishing the pathological behavior of the isolated virus when compared to a human immunodeficiency virus not having at least one such mutation.

The isolated virus may contain additional mutations. The additional mutations may also be involved in the delaying or diminishing of the pathological behavior of the isolated virus when compared to a human immunodeficiency virus not having at least one such mutation. The additional mutation may render the isolated virus even more attenuated. Thus, in another embodiment, the invention provides a method for obtaining the virus according to the invention comprising collecting a certain number of strains, sequencing at least part of the strains, comparing obtained sequences with sequences of virus according to the invention, and isolating strains comprising sequence similarities to the virus according to the invention. In another embodiment, the strain is amplified before sequencing in the method.

A method according to the invention is particularly useful for obtaining an attenuated virus according to the invention. Therefore, in another embodiment, the invention provides a virus obtainable by a method according to the invention.

The virus according to the invention may be used to prepare a vaccine. If administered to an immunocompetent individual, the individual will develop antibodies against human immunodeficiency virus. The antibodies give the individual at least partial protection against more virulent strains. Thus, the invention provides the virus according to the invention for use as a vaccine. As the strains that we have isolated thus far are still capable of reducing the number of CD4+ T cells in an individual infected with the strains, the virus according to the invention is preferably processed further. In combination with other changes in the human immunodeficiency virus genome, the mutations described in Tables 1 through 4, or a selection thereof, can be used for the design of a safe live attenuated human immunodeficiency virus vaccine. In addition, the same mutations can be used in vaccines composed of dead virus or virus without replicatable nucleic acid or protein subunits. These mutations have been shown to be immunogenic and to provoke an immune response capable of suppressing the growth of the human immunodeficiency virus. In part, this will be the result of features of the individual's immune system, but another equally essential factor is the attenuated human immunodeficiency virus itself. The immunogenic determinants of the proteins play a central role in the quality and characteristics of the evoked immune response.

Thus, another embodiment of the present invention provides the use of the virus according to the invention for the preparation of a vaccine. Of course, the vaccine will specifically provide an individual with at least partial protection against AIDS. Thus, the invention provides a use of the virus according to the invention for the preparation of a vaccine for AIDS. In yet another embodiment, the invention provides a vaccine comprising the virus according to the invention. The vaccine of the invention is particularly useful for prophylaxis against AIDS. Therefore, the present invention provides a method for at least partial prophylaxis against AIDS, comprising administering the vaccine according to the invention to an individual.

With the teachings of the present invention, a person of ordinary skill in the art is capable of identifying the virus according to the invention in an individual. Mutations comprised by the virus according to the invention can be used as target sequences for diagnostic assays to discriminate human immunodeficiency virus sequences with and without the mutations described in Tables 1 through 4. Diagnostics capable of identifying these mutations may play a role in assessing the life expectancy of infected individuals because these mutations, or a subset thereof, indicate a better quality of life and a longer disease-free period compared to other human immunodeficiency virus strains. Therefore, another embodiment of the invention provides a method for identifying the virus according to the invention in an individual, comprising collecting a sample containing virus, or parts thereof, from the individual and detecting strains comprising sequence similarities to the virus according to the invention. Preferably, the sample is a plasma, serum, or blood sample. Virus may be collected from an individual by collecting blood samples comprising peripheral blood monocytic cells (PBMC). Thus, another embodiment of the invention provides a method wherein the virus is collected by isolating peripheral blood monocytic cells from the individual.

Sequence similarities are defined as previously set forth. A person ordinarily skilled in the art is able to determine sequence similarities. For instance, an ordinarily skilled artisan is able to detect the virus according to the invention using antibodies with a binding specificity for one or more of the stable mutations of the virus. Alternatively, a person of ordinary skill in the art can detect sequence similarities by sequencing collected virus from an individual. Sequencing techniques are well known in the art. Thus, another embodiment of the invention provides a method wherein the sequence similarities are detected by sequencing.

Of course, other techniques are available to detect sequence similarities between an isolated strain and the virus of the invention. One non-limiting example of an alternative to sequencing is hybridization with probes comprising at least one sequence of the virus according to the invention. Thus, yet another embodiment of the invention provides a method wherein the sequence similarities are detected by hybridization with probes comprising at least one sequence of the virus according to the invention. Those ordinarily skilled in the art can think of other techniques to detect sequence similarities between an isolated strain and the virus according to the invention. The scope of the present invention therefore includes all techniques for detection of sequence similarities.

The following examples of the present invention are given by way of illustration only. They are not to be construed as limiting the invention in any way. Those of ordinary skill in the art can perform alternative experiments, which alternatives remain within the scope of the present invention.

### EXAMPLES

### Example 1

In this example we describe the sequencing of full genome sequences of HIV-1. The method comprises the following steps.

1. Preparation of Generic Amplification Tool (GAT) Mixtures:

Mix A

| Solution | 1x (μl) |
|---|---|
| 10x PCR buffer II | 2 |
| 100 mM MgCl₂ | 1 |
| 100 μM dNTP's | 0.8 |
| 100 ng/μl oligo JZH2R | 0.5 |
| H₂O | 4.7 |
| RNAsin** | 0.5 |
| 50 U/μl MuLV-RT* | 0.5 |

*Add the enzyme before use.
**For DNA sequencing, no RNAsin in mixture.
10x PCR buffer II (500 mM KCl, 100 mM Tris-HCl, pH8.3; included in Perkin Elmer kit N808-0161).
RNAsin (Perkin Elmer N808-0119).
MuLV-RT (Perkin Elmer N808-0018).
JZH2R primer: 5′- GCT ATC ATC ACA ATG GAC NNN NNG - 3′ (SEQ ID NO: 2).

Mix B

| Solution | 1x (μl) |
|---|---|
| 10x seq. buffer2 | 4 |
| 100 μm dNTP's | 1.6 |
| 100 ng/μl oligo JZH2R | 1 |
| H₂O | 13.2 |
| 13 U/μl Sequenase 2.0* | 0.2 |

*Add the enzyme just before use.
10x seq. buffer2 (350 mM Tris-HCl pH7.5, 175 mM MgCl₂, 250 mM NaCl).
Sequenase 2.0 (Amersham, USB 70775).

PCR mix

| Solution | 1x (μl) |
|---|---|
| 10X PCR buffer | 5.0 |
| 100 mM MgCl₂ | 0.9 |
| 100 μM dNTP's | 0.2 |
| 100 ng/μl JZH1 | 1.0 |
| H₂O | 40.6 |
| 5 U/μl Amplitaq | 0.3 |

Amplitaq (Perkin Elmer N808-0161).
JZH1 primer: 5′ - GCT ATC ATC ACA ATG GAG - 3′ (SEQ ID NO: 3).

2. Isolation of Nucleic Acid

10 μl of culture supernatant is used to isolate the nucleic acid with Protocol Y described by Boom et al., J. Clin. Microbiol. 1990 March; 28(3):495-503.

elute the nucleic acid with 30 μl H₂O.

3. GAT

1. First Strand Synthesis*

Take 10 μl of PROTOCOL Y/Sc¹ isolated product.

Incubate 5 minutes at 80° C., quench on ice.

Add 10 μl mix A (JZH2R; MuLV-RT, add enzyme before use).

Incubate 10 minutes at room temperature.

Incubate 30 minutes at 42° C.

Incubate 5 minutes at 80° C., subsequently cool down to room temperature.

Add 0.5 μl RNAse-H (1U/μl; Boehringer Mannheim, 786357).

Incubate 30 minutes at 37° C.

2. Second Strand Synthesis

Take 20 μl of the first strand synthesis (keep it on ice).

Add 20 μl of mix B (JZH2R; Sequenase 2.0, add enzyme before use).

Incubate 10 minutes on ice.

Incubate 10 minutes at room temperature.

Incubate 30 minutes at 37° C.

Store either on ice for following amplification or in the −80 for later use. It is best to perform the PCR immediately after the first and second synthesis.

2 μl of product is used for PCR.

3. PCR*

48 μl PCR-mix (JZH1).

Add 2 μl of GAT product.

4. PCR Program (Perkin Elmer 9700 PCR Machine).

5 minutes at 95° C.

20 seconds at 95° C., 30 seconds at 55° C., 2 minutes at 72° C. for 45 cycles.

10 minutes at 72° C.

10 minutes at 4° C.

15 μl was examined on 1.2% agarose gel and the method was considered to be successful if long smears could be observed in the gel.

Dilute GAT product for multiple specific HIV-1 PCR reactions. Standard dilution rate as input for the amplification is 10 times (10 μl GAT product +90 μl Baker water) or 100 times (10 μl GAT product +990 μl Baker water). A dilution rate of 100 times usually generates the best results. Therefore, the 100 times dilution is used for amplification first. If the result is not satisfactory, an additional amplification using the 10 times dilution is done.

Subsequently, perform 20 specific HIV-1 PCR reactions (see list for primer sets and details) according to standard PCR amplification specifications.

| 5′ PRIMER | 3′ PRIMER | | DETAILS |
|---|---|---|---|
| 5′ PROT1 | 3′ PROTII | 572 | PROT FM |
| 5′ V3NOT | 3′ ENV KN | 1628 | L2 |
| 5′ V3NOT | 3′ ENV KN | 1628 | 3′ ENV KN |
| 5′ V3NOT | 3′ ENV KN | 1628 | L3 |
| 5′ V3NOT | 3′ ENV KN | 1600 | WS9rev |
| 5′ V3NOT | 3′ ENV KN | 1600 | GP12O 3′ PCR |
| 5′ V3NOT | L9 | 900 | L9 |
| 5′ V3NOT | L9 | 900 | 5′ V3NOT |
| FGS0001 | FGS0002 | 670 | FGS002 |
| FGS0001 | FGS0002 | 670 | FGS001 |
| FGS0003 | FGS0004 | 688 | FGS004 |
| FGS0003 | FGS0004 | 688 | FGS003 |
| FGS0005 | FGS0006 | 535 | FGS006 |
| FGS0005 | FGS0006 | 535 | FGS005 |
| FGS0005 | FGS0008 | 1095 | FGS007 |
| LOUW-1-GAG | SK39 | 900 | LOUW-1-GAG |
| LOUW-1-GAG | SK39 | 900 | SK431 |
| LOUW-1-GAG | SK39 | 900 | GAGAE-3T7 |
| POL 5′ FM | VPR3-T7 | 1050 | POL5′ FM |
| POL 5′ FM | VPR3-T7 | 1050 | VPR3-T7 |
| PROT FM | 3′ HALFRT | 1500 | SP6 P66 |
| PROT FM | 3′ HALFRT | 1500 | ENDPROTT7 |
| PROT FM | 3′ HALFRT | 1500 | 3′ HALFRT |
| PROT FM | ENDPROTT7 | 1000 | PROT FM |
| RT19new | 3′ HALFRT | 1045 | ENDPROTT7 |
| RT19new | 3′ HALFRT | 1045 | 3′ HALFRT |
| RT19new | ET08 | 647 | RT19new |
| SK102 | P6END | 955 | P6END |
| SK102 | T7P6PROT | 950 | SK102 |
| SP6 P66 | 3′ HALFRT | 600 | ET43 |
| SP6 P66 | ET19 | 1312 | ET43 |
| SP6 P66 | ET19 | 1312 | ET19 |
| V1V2-1 | 3′ KST-TT7 | 850 | V1V2-2 SP6 |
| VPR-1 | V1V2-4 | 1440 | VPU-4 |
| VPR-1 | V1V2-4 | 1440 | VPR-1 |
| VPR-1 | V1V2-4 | 1440 | VPU-1 |
| VPR-1 | V1V2-4 | 1440 | L8 |
| VPR-1 | V1V2-4 | 1440 | V1V2-4 |
| VPR-1 | V1V2-4 | 1440 | VPR-4 |
| WS9REV | FGS014 | 1491 | 3′ TAT-1 |

PCR mix.

| Solution | 1x (μl) |
|---|---|
| 10X PCR buffer | 5.0 |
| 100 mM MgCl₂ | 1.0 |
| 100 μM dNTP's | 0.4 |
| 100 ng/μl 5′ PRIMER | 0.5 |
| 100 ng/μL 3′ PRIMER | 0.5 |
| H₂O | 37.6 |
| 5 U/μl Amplitaq | 0.2 |

Add 5 μl of diluted product from the GAT method.

PCR Program (Perkin Elmer 9700 PCR Machine).

5 minutes at 95° C.

1 minute at 95° C., 1 minute at 55° C., 2 minutes at 72° C. for 35 cycles.

10 minutes at 72° C.

10 minutes at 4° C.

5 μl is examined on 1.0% agarose gel and length of the PCR fragments is checked in comparison with a length marker run on the same gel.

Subsequently, all PCR fragments were sequenced according to the Bigdye sequencing protocol (Applied Biosystems) using at least the following set of sequence primers.

| Primer | Sequence | | | Gene ID |
|---|---|---|---|---|
| 3′P6END | TAA TAC TGT ATC ATC TGC TCC T | (SEQ ID NO:4) | 3′ | GAG |
| 3′T7P6 PROT | TAA TAC GAC TCA CTA TAG GGT ACT GTG ACA AGG GGT CGT TGC CA | (SEQ ID NO:5) | 3′ | GAG |
| LOUW-1-GAG | TTG ACT AGC GGA GGC TAG AA | (SEQ ID NO:6) | 5′ | GAG |
| VIV2-1 | TGT GTA CCC ACA GAC CCC AAC CC | (SEQ ID NO:7) | 5′ | V1V2 |
| V1V2-2SP6 | ATT TAG GTG ACA CTA TAG GAG GAT ATA ATC AGT TTA TGG GA | (SEQ ID NO:8) | 5′ | V1V2 |
| VIV2-4 | ATT CCA TGT GTA CAT TGT ACT G | (SEQ ID NO:9) | 3′ | V1V2 |
| 5′V3NOT | GCG CGG CCG CAC AGT ACA ATG TAC ACA TGG | (SEQ ID NO:10) | 5′ | V3 |
| KSIT7 | TAA TAC GAC TCA CTA TAG GGT GGG TCC CCT CCT GAG GA | (SEQ ID NO:11) | 3′ | V3 |
| SK39 | TTT GGT CCT TGT CTT ATG TCC AGA ATG C | (SEQ ID NO:12) | 3′ | GAG |
| SK102 | GAG ACC ATC AAT GAG GAA GCT GCA GAA TGG GAT | (SEQ ID NO:13) | 5′ | GAG |
| SK 431 | TGC TAT GTC AGT TCC CCT TGG TTC TCT | (SEQ ID NO:14) | 3′ | GAG |
| PROT-FM | CAA GGG AAG GCC AGG GAA TTT | (SEQ ID NO:15) | 5′ | POL |
| SP6P66 | GAT TTA GGT GAC ACT ATA GAG ATA TCA GTA CAA TGT GCT | (SEQ ID NO:16) | 5′ | GAG |
| HALFRT | TAT TTC TGC TAT TAA GTC TTT TGA TGG GTC A | (SEQ ID NO:17) | 3′ | RT |
| ENDPROTT7 | TAA TAC GAC TCA CTA TAG GGA ATA TTG CTG GTG ATC CTT TCC A | (SEQ ID NO:18) | 3′ | POL |
| GAGAE-3T7 | TAA TAC GAC TCA CTA TAG GGA CTA TTT TAT TTA ATC CCA GGA T | (SEQ ID NO:19) | 3′ | GAG |
| VPR-1 | GAT CTC TAC ATT ACT TGG CAC T | (SEQ ID NO:20) | 5′ | VPR |
| VPR3-T7 | TAA TAC GAC TCA CTA TAG GGA AAG CAA CAC TTT TTA CAA TAG CA | (SEQ ID NO:21) | 3′ | VPR |
| VPR-4 | CTT CTT CCT GCC ATA GGA GAT GCC | (SEQ ID NO:22) | 3′ | VPR |
| VPU-1 | GCA TCT CCT ATG GCA GGA AGA AG | (SEQ ID NO:23) | 5′ | VPU |
| VPU-4 | ATA TGC TTT AGC ATC TGA TGC ACA AAA TA | (SEQ ID NO:24) | 3′ | VPU |
| POL5′FM | TGG AAA GGA CCA GCA AAG CTC CTC TGG AAA GGT | (SEQ ID NO:25) | 5′ | POL |
| L9 | CCC AAG GAA CAA AGC TCC | (SEQ ID NO:26) | 3′ | ENV |
| FGS001 | GTT AGT GGG AAA ATT GAA TTG GGC A | (SEQ ID NO:27) | 5′ | RT |
| FGS002 | AAA TTG CTT GTA ACT CAG TCT TCT | (SEQ ID NO:28) | 3′ | RT |
| FGS003 | ATG GGG CAG CTA ACA GGG AGA CTA | (SEQ ID NO:29) | 5′ | RT |
| FGS004 | TGT TTT TAC TGG CCA TCT TCC TGCT | (SEQ ID NO:30) | 3′ | RT |
| FGS005 | GGT AGC AGT TCA TGT AGC CAG TGG A | (SEQ ID NO:31) | 5′ | RT |
| FGS006 | CTT GTA TTA CTA CTG CCC CTT CAC CT | (SEQ ID NO:32) | 3′ | RT |
| L8 | AGA GCA GAA GAC AGT GGC | (SEQ ID NO:33) | 5′ | VPU |
| L3 | GGA GCA GCA GGA AGC ACT ATG | (SEQ ID NO:34) | 5′ | ENV |
| L2 | TAG GTA TCT TTC CAC AGC CAG | (SEQ ID NO:35) | 3′ | ENV |
| FGS007 | CTA ATC CTC ATC CTG TCT ACT | (SEQ ID NO:36) | 5′ | RT |
| FGS008 | AGT TTC GTA ACA CTA GGC AAA GGT | (SEQ ID NO:37) | 3′ | RT |
| WS9REV | TAT TAA CAA GAG ATG GTG GT | (SEQ ID NO:38) | 5′ | ENV |
| GP120 3′PCR | GCT CCC AAG AAC CCA AGG AA | (SEQ ID NO:39) | 3′ | ENV |
| RT 19NEW | CAC CTG TCA ACA TAA TTG GAA G | (SEQ ID NO:40) | 5′ | RT |
| FGS014 | CTT TTA AAA AGT GGC TAA GAT CT | (SEQ ID NO:41) | 3′ | ENV |
| 3TAT-1 | TTT GAA TTC TAA TCG AAT GGA TCT GTC TC | (SEQ ID NO:42) | 3′ | ENV |
| ET19 | GAT ATT TCT CAT GTT CAT CTT GGG CCT TAT CTA TTC C | (SEQ ID NO:43) | 3′ | RT |

Sequences that were obtained were subsequently edited and assembled by AUTOASSEMBLER software. Before starting AUTOASSEMBLER, the sequences are edited with basic sequence analysis software in order to organize and check the raw data. The edited sequences are loaded into AUTOASSEMBLER. After assemblage in AUTOASSEMBLER, a CONTIG is formed. This CONTIG is subsequently checked for mistakes. If a part of the sequence is not clear, additional experiments have to be done. All software used is supplied by Applied Biosystems.

### Example 2

In this example we isolated PBMC (peripheral blood monocytic cells) from an HIV-1 infected individual and isolated HIV-1 biological clones from these cells. PBMC were obtained from heparinized venous blood by isolation on a Percoll gradient. PBMC were suspended in Iscove's modified Dulbecco's medium supplemented with 10% DMSO, 20% fetal calf serum and antibiotics (penicillin (100 U/ml) and streptomycin (100 μg/ml)). Cells were suspended at concentrations of approximately 5×10⁶ cells/ml and aliquots of 1 ml were viably frozen and stored in liquid nitrogen until use. Cryopreserved PBMC were thawed and washed with culture medium (Iscove's modified Dulbecco's medium supplemented with 10% fetal calf serum, recombinant interleukin-2 (20 U/ml, PROLEUKIN; Chiron Benelux BV) and antibiotics (penicillin (100 U/ml) and streptomycin (100 μg/ml)) to remove residual DMSO. In a 96-well plate, serial dilutions of HIV-1 infected PBMC (0.5×10⁴ to 4×10⁴ per well) were cocultivated with 2 to 3 days phytohaemagglutinin (PHA) stimulated healthy donor PBMC (1×10⁵ per well) in a final volume of 200 μl culture medium for 28 days. For each cell dilution, multiple cocultures (28 wells) were performed. At days 7, 14, and 21, half of the culture supernatants was harvested for analysis of viral p24 production using an in-house antigen capture ELISA. Cells were resuspended and were transferred to 96-well plates containing fresh healthy donor PHA-stimulated PBMC (1×10⁵ per well) and further cultured in a volume of 200 μl. From wells with cultures positive for p24 antigen, virus stocks were grown in 25 ml culture flasks. Cell free supernatants of these viral cultures were aliquotted and stored at −70° C. Viruses obtained using this procedure were considered to be clonal if less than one third of the wells of a cell dilution were positive for p24.

### REFERENCES

1. Berkhout B. Verhoef K, van Wamel J, Back B. 1999. Genetic instability of live-attenuated HIV-1 vaccine strains. J. Virol. 73: 1138-1145.

2. Baba T W, Liska V, Khimani A H, Ray N B, Dailey P J, Penninck D, Bronson R. Greene M F, McClure H M, Martin L N, 1999. Live attenuated, multiply deleted simian immunodeficiency virus causes Aids in infant and adult macaques. Nature Medicine 5: 194-203.

3. Dyer W B, Ogg G S, Demoitie M-A, Jin X, Geczy A F, Rowland-Jones S L, McMichael A J, Nixon D F, Sullivan J S. 1999. Strong human immunodeficiency virus (HIV)-specific cytotoxic T-lymphocyte activity in Sydney blood bank cohort patients infected with nef-defective HIV type 1. J. Virol. 73: 436-443.

4. Greenough T C, Sullivan J L, Desrosiers R C. 1999. Declining CD4 T-cell counts in a person infected with nef-deleted HIV-1. New Engl. J. Med. 340: 236-237.

5. Boom R, Sol C J, Salimans M M, Jansen C L, Wertheim-van Dillen P M, van der Noordaa J. Rapid and simple method for purification of nucleic acids. J. Clin. Microbiol. 1990 March; 28(3):495-503.

TABLE 1
Unique polymorphisms in the HIV-1 sequences isolated from patient
671 that are present in the whole period of infection.

| Region | Amino acid | Polymorphic | Consensus |
|---|---|---|---|
| of HIV | number | amino acid sequences | B |
| Gag | 118 | 3 aa (QAE) insertion | |
| | 464 | 10 aa (QSRPEPTAPP) duplication | |
| | 541 | stop codon² | S |
| Pol | 196 (RT 41) | L | M |
| | 370 (RT 215) | Y³, D | T |
| | 621-624 | IPIK | VSLN/T |
| Nef | 48-49 | 2 aa deletion⁴ | |
| Env | 423-425 | LYK | QFC |
| ¹Amino acid numbering is according to the numbering of the amino acid sequences of the HIV-1 consensus B sequences of the different HIV-1 genes in the Los Alamos database (http://hiv-web.lanl.gov, Human Retroviruses and AIDS 1999: A Compilation and Analysis of Nucleic Acid and Amino Acid Sequences. Kuiken C L, Foley B, Hahn B, Korber B. McCutchan F, Marx P A, Mellors J W, Mullins J I, Sodroski J, and Wolinksy S, Eds. | | | |
| # Theoretical Biology and Biophysics Group, Los Alamos National Laboratory, Los Alamos, NM). | | | |

²Two amino acids before the normal stop codon of gag.
³AZT resistance conferring mutations.
⁴Polymorphism previously described by Alexander et al. (2000) J. Virol 74.

TABLE 2
Genotypic characteristics of the HIV-1 sequences at early versus late
time point isolations in the infection.

| Region of HIV | Amino acid number | Early isolation | Late isolation |
|---|---|---|---|
| Pol (p66/p51) | 60 | V | I |
| | 135 | I | T |
| | 215 | Y | D |
| | 593 | I | V |
| Rev | 51 | Q | R |
| | 54 | S | A |
| Nef | 9 | S | K |
| | 113 | I | V |
| Tat | 52 | W | R |
| | 100 | V | G |
| | 43 | G | E |
| | 117 | N | D |
| | 159 | E | A |
| | 187 | K | S |
| | 193 | V | I |
| Env | 201 | R | K |
| gp120 | 207 | Q | K |
| | 243 | R | G |
| | 343 | T | A |
| | 350 | R | K |
| | 416 | I | G |
| | 439 | P | S |
| | 456 | G | R |
| gp41 | 505 | S | P |
| 1. Amino acid numbering is according to the numbering of the amino acid sequences of the HIV-1 consensus B sequences of the different HIV-1 genes in the Los Alamos database (http://hiv-web.lanl.gov, Human Retroviruses and AIDS 1999: A compilation and Analysis of Nucleic Acid and Amino Acid Sequences. Kuiken C L, Foley B, Hahn B, Korber B, McCutchan F, Marx P A, Mellors J W, Mullins J I, Sodroski J, and Wolinksy S, Eds. Theoretical Biology and Biophysics Group, Los | | | |
| # Alamos National Laboratory, Los Alamos, NM). | | | |

TABLE 3
Genotypic characteristics of the HIV-1 sequences isolated late in the
infection that replicate fast versus slow in culture.

| | Amino Acid | Fast | Slow |
|---|---|---|---|
| Region of HIV | Number | Replication | Replication |
| Pol (p66/p51) | 40 | E | A |
| | 841 | G | D |
| Rev | 18 | L | I |
| | 21 | F | Y |
| | 84 | G | D |
| Gag | 385 | N | S |
| | 538 | N | S |
| Vif | 9 | G | I |
| | 17 | H | Y |
| | 61 | D | E |
| | 130 | R | S |
| | 159 | K | Q |
| Vpr | 41 | G | V |
| Env | 4 | R | K |
| gp120 | 31 | M | T |
| | 44 | K | N |
| | 193 | T | A |
| | 329 | E | D |
| gp41 | 530 | E | A |
| | 736 | N | E |
| | 752 | N | S |
| | 845 | G | E |
| | 891 | V | I |
| | 937 | L | F |
| 1. Amino acid numbering is according to the numbering of the amino acid sequences of the HIV-1 consensus B sequences of the different HIV-1 genes in the Los Alamos database (http://hiv-web.lanl.gov, Human Retroviruses and AIDS 1999: A Compilation and Analysis of Nucleic Acid and Amino Acid Sequences. Kuiken C L, Foley B, Hahn B, Korber B, McCutchan F, Marx P A, Mellors J W, Mullins J I, Sodroski I, and Wolinksy S, Eds. Theoretical Biology and Biophysics Group, Los | | | |
| # Alamos National Laboratory, Los Alamos, NM). | | | |

TABLE 4
Genotypic characteristics of the HIV-1 sequences isolated early in the
infection that replicate fast versus slow in culture.

| | Amino Acid | Fast | Slow |
|---|---|---|---|
| Region of HIV | Number | Replication | Replication |
| Rev | 52 | I | Q |
| | 111 | E | D |
| Nef | 101 | L | F |
| | 197 | H | Q |
| | 207 | F | Y |
| Tat | 11 | C | W |
| | 64 | N | T |
| | 85 | E | K |
| Env | 4 | K | R |
| gp120 | 171 | I | T |
| | 172 | T | S |
| | 392 | I | T |
| gp41 | 396 | R | I |
| | 860 | S | G |
| | 910 | D | E |
| | 926 | 5 | I |
| 1. Amino acid numbering is according to the numbering of the amino acid sequences of the HIV-1 consensus B sequences of the different HIV-1 genes in the Los Alamos database (http://hiv-web.lanl.gov, Human Retroviruses and AIDS 1999: A compilation and Analysis of Nucleic Acid and Amino Acid Sequences. Kuiken C L, Foley B, Hahn B, Korber B, McCutchan F, Marx P A, Mellors J W, Mullins J I, Sodroski J, and Wolinksy S, Eds. Theoretical Biology and Biophysics Group, Los | | | |
| # Alamos National Laboratory, Los Alamos, NM). | | | |

What is claimed is:

1. An isolated human immunodeficiency virus, comprising at least one non-revertant mutation capable of delaying or diminishing the pathological behavior of said human immunodeficiency virus as compared with a human immunodeficiency virus not having at least one such mutation.

2. The isolated human immunodeficiency virus of claim 1, wherein said at least one non-revertant mutation is located in the gag gene or the pol gene.

3. The isolated human immunodeficiency virus of claim 1, wherein said at least one non-revertant mutation comprises at least one mutation selected from the group consisting of:
an insertion of the amino acid sequence Gln-Ala-Glu at amino acid number 118 in the gag region,
an insertion of the amino acid sequence Gln-Ser-Arg-Pro-Glu-Pro-Thr-Ala-Pro-Pro (SEQ ID NO: 1) at amino acid number 464 in the gag region,
a stop codon two amino acids before the normal stop codon of the gag region,
a substitution of Leu for Met at amino acid number 196 (RT 41) in the pol region,
a substitution of Tyr or Asp for Thr at amino acid number 370 (RT 215) in the pol region,
a substitution of the amino acid sequence Ile-Pro-ILe-Lys for the amino acid sequence Val-Ser-Leu-Asn/Thr at amino acid numbers 621-624 in the pol region,
a deletion of amino acids 48-49 in the nef region, and
a substitution of the amino acid sequence Leu-Tyr-Lys for the amino acid sequence Gln-Phe-Gly at amino acids 423-425 in the env region.

4. A method for obtaining the isolated human immunodeficiency virus of claim 1, comprising providing a wild-type human immunodeficiency virus with at least one non-revertant mutation capable of delaying or diminishing the pathological behavior of said isolated human immunodeficiency virus as compared with a human immunodeficiency virus not having at least one such mutation.

5. A method for obtaining the isolated human immunodeficiency virus of claim 1, comprising:
collecting a number of strains,
sequencing at least part of each of said number of strains,
comparing obtained sequences with sequences of said isolated human immunodeficiency virus, and
isolating strains comprising sequence similarities to said isolated human immunodeficiency virus.

6. A method for obtaining the isolated human immunodeficiency virus of claim 2, comprising:
collecting a number of strains,
sequencing at least part of each of said number of strains,
comparing obtained sequences with sequences of said isolated human immunodeficiency virus, and
isolating strains comprising sequence similarities to said isolated human immunodeficiency virus.

7. A method for obtaining the isolated human immunodeficiency virus of claim 3, comprising:
collecting a number of strains,
sequencing at least part of each of said number of strains,
comparing obtained sequences with sequences of said isolated human immunodeficiency virus, and
isolating strains comprising sequence similarities to said isolated human immunodeficiency virus.

8. The method according to claim 5, wherein said number of strains are amplified before sequencing.

9. The method according to claim 6, wherein said number of strains are amplified before sequencing.

10. The method according to claim 7, wherein said number of strains are amplified before sequencing.

11. A vaccine comprising the isolated human immunodeficiency virus of claim 1.

12. A vaccine comprising the isolated human immunodeficiency virus of claim 2.

13. A vaccine comprising the isolated human immunodeficiency virus of claim 3.

14. A method for identifying the isolated human immunodeficiency virus of claim 1, comprising:
collecting a sample comprising human immunodeficiency virus or parts thereof from an individual, and
detecting collected human immunodeficiency virus strains comprising sequence similarities to said isolated human immunodeficiency virus.

15. A method for identifying the isolated human immunodeficiency virus of claim 2, comprising:
collecting a sample comprising human immunodeficiency virus or parts thereof from an individual, and
detecting collected human immunodeficiency virus strains comprising sequence similarities to said isolated human immunodeficiency virus.

16. A method for identifying the isolated human immunodeficiency virus of claim 3, comprising:
collecting a sample comprising human immunodeficiency virus or parts thereof from an individual, and
detecting collected human immunodeficiency virus strains comprising sequence similarities to said isolated human immunodeficiency virus.

17. The method according to claim 14, wherein said sample comprising human immunodeficiency virus or parts thereof is collected by isolating peripheral blood monocytic cells from said individual.

18. The method according to claim 15, wherein said sample comprising human immunodeficiency virus or parts thereof is collected by isolating peripheral blood monocytic cells from said individual.

19. The method according to claim 16, wherein said sample comprising human immunodeficiency virus or parts thereof is collected by isolating peripheral blood monocytic cells from said individual.

20. The method according to claim 14, wherein said sequence similarities are detected by sequencing.

21. The method according to claim 15, wherein said sequence similarities are detected by sequencing.

22. The method according to claim 16, wherein said sequence similarities are detected by sequencing.

23. The method according to claim 14, wherein said sequence similarities are detected by hybridization with probes comprising at least one sequence of said isolated human immunodeficiency virus.

24. The method according to claim 15, wherein said sequence similarities are detected by hybridization with probes comprising at least one sequence of said isolated human immunodeficiency virus.

25. The method according to claim 16, wherein said sequence similarities are detected by hybridization with probes comprising at least one sequence of said isolated human immunodeficiency virus.

26. A method for providing at least partial prophylaxis against acquired immunodeficiency syndrome, comprising administering the vaccine of claim 11.

27. A method for providing at least partial prophylaxis against acquired immunodeficiency syndrome, comprising administering the vaccine according to claim 12.

28. A method for providing at least partial prophylaxis against acquired immunodeficiency syndrome, comprising administering the vaccine according to claim 13.
